(19) United States
(12) Patent Application Publication
(10) Pub. No.: US 2014/0358009 A1
(43) Pub. Date: Dec. 4, 2014

(54) System and Method for Collecting Eye-Movement Data

(71) Applicants: Michael O'Leary, Del Mar, CA (US); Ian Purcell, San Diego, CA (US); Yinhong Qu, Tianjin (CN)

(72) Inventors: Michael O'Leary, Del Mar, CA (US); Ian Purcell, San Diego, CA (US); Yinhong Qu, Tianjin (CN)

(21) Appl. No.: 13/906,172

(22) Filed: May 30, 2013

(51) Int. Cl. A61B 5/00; A61B 3/00; A61B 3/14; A61B 3/113; A61B 5/11

(52) U.S. Cl. CPC A61B 5/4023; A61B 3/113; A61B 5/1128; A61B 5/0077; A61B 5/6803; A61B 3/14; A61B 3/0025; A61B 3/0083; A61B 5/0013
USPC 600/476; 600/558

(57) ABSTRACT

A system for creating an eye-movement record that is useful for diagnosing balance disorders of a patient includes a head-mounted unit having an indicator which generates head position signals. Also included is a computer/controller for progressing the patient through a sequence of predetermined head orientations. An imaging unit in the head-mounted unit is used to create an eye-movement record for each head orientation, and the records are then archived in a recorder. With instructions from the patient, a de-identified eye-movement record can be sent to selected regional specialists for further evaluation and possible treatment.

### FIELD OF THE INVENTION

The present invention pertains generally to systems and methods for diagnosing balance disorders. More particularly, the present invention pertains to head-mounted diagnostic units that can be individually used for generating eye-movement records in an outpatient environment. The present invention is particularly, but not exclusively, useful as a system and method for creating confidential eye-movement records that are maintained and controlled by the person creating the record.

### BACKGROUND OF THE INVENTION

Vertigo (i.e. dizziness) is a symptom of a balance disorder that can result for any of various reasons. Anatomically, vertigo results when crystals in the inner ear of an individual are erratically misaligned. In some instances, vertigo may be only temporary, such as when the pilot of an aircraft becomes spatially disoriented while flying on instruments. Vertigo, however, can also be pathological. In any event, it is known that a patient will experience erratic eye-movements during vertigo. For diagnostic purposes, it is known that properly stimulated eye movements can be evaluated to determine whether pathological conditions exist. More specifically, it is well known that a properly trained specialist (physician) can evaluate the results of a Dix-Hallpike Maneuver, or a recognized equivalent of this maneuver, and thereby diagnose the nature and extent of a balance disorder.

Heretofore, some degree of supervision has been required for the conduct of a Dix-Hallpike Maneuver. Always, it has been, and still is, necessary for a trained specialist to make a proper diagnosis. Nevertheless, the conduct of a Dix-Hallpike Maneuver requires no medical intervention, and it can be accomplished merely by prompting the patient through a sequence of predetermined head orientations. Images of eye movements taken in the different head orientations can then be evaluated and used to diagnose any balance disorder.

As noted above, vertigo may result for many various reasons. And, for many different reasons, a patient may want to maintain confidentiality for his/her condition (balance disorder). Accordingly, it is desirable there be a system that is available for use by a patient, either alone or with proper supervision, to obtain a reliable diagnosis from a trained specialist under confidential circumstances in an outpatient status.

With the above in mind, it is an object of the present invention to provide a system for collecting eye-movement data to create an eye-movement record that can be used as a diagnostic of balance disorders, wherein the eye-movements are responsive to a sequence of changes in head orientation, and wherein a required sequence of head orientations are prompted by computer control. Another object of the present invention is to provide a system for creating eye-movement records wherein the required sequence of head orientations can be performed individually without assistant supervision. Still another object of the present invention is to provide a system for creating confidential eye-movement records that can be archived and controlled by the patient for subsequent evaluation and possible treatment. Yet another object of the present invention is to provide a system for creating eye-movement records that is simple to use, is easy to assemble, and is comparatively cost effective.

### SUMMARY OF THE INVENTION

In accordance with the present invention, a system and method are provided for collecting eye-movement data that can be used to create a plurality of eye-movement records. Collectively, the plurality of eye-movement records is then used as a diagnostic for evaluating balance disorders. For this purpose, a patient's eye-movements are responsive to the sequence of changes in head orientation prescribed for a Dix-Hallpike Maneuver. Importantly, as envisioned for the present invention, the eye-movement data can be generated individually by the patient, without assistant supervision, while in an outpatient status. Also, the resultant eye-movement records can be maintained and confidentially controlled by the patient.

Structurally, the system of the present invention includes goggles that can be positioned and worn on the head of the patient. An indicator is mounted on the goggles and is to be moved with the head of the patient. In particular, the indicator is incorporated to generate position signals that are respectively indicative of each predetermined head orientation required by the Dix-Hallpike Maneuver. Specifically, the Dix-Hallpike Maneuver consists of head movements through a sequence of seven separate head orientations.

In order, these orientations are:

1. patient sits with head centered;

2. patient sits with head 45 degrees to the right;

3. patient lays back with head 45 degrees to the right and held in approximately 20 degrees of extension;

4. patient sits back up with head centered;

5. patient sits with head 45 degrees to the left;

6. patient lays back with head 45 degrees to the left and held in approximately 20 degrees of extension; and

7. patient sits back up with head centered.

Along with the indicator, an imaging unit with a camera is also mounted on the goggles. Like the indicator, the imaging unit is intended to move with the head of the patient to create eye-movement records. Further, the system includes a recorder that is connected to the imaging unit to archive the eye-movement records as they are created by the imaging unit.

A computer/controller for operating the system is connected to the indicator, to the imaging unit, and to the recorder. Also, the computer/controller is externally connected with a regional data base that contains respectively pertinent information on a plurality of specialists. In this combination, a central server is incorporated with the recorder (i.e. the computer/controller) for forwarding confidential eye-movement records to the data base for subsequent use by at least one selected specialist in response to instructions from the patient.

In an operation of the system of the present invention, the patient positions the goggles on his/her head. The computer/controller then notifies the patient that an operation of the system will begin when his/her head is moved into the starting orientation required by the Dix-Hallpike Maneuver. In response to a notification from the computer/controller that the head of the patient is being properly held in the predetermined orientation, the patient presses on a “go” button. This activates the imaging unit. Specifically, the camera of the imaging unit then creates an eye-movement record while the head of the patient is held substantially stationary in the particular orientation for a preselected time interval (e.g. less than about ten seconds). Once an eye-movement record has been created at one predetermined orientation, the computer/controller then notifies the patient to move his/her head to the next sequentially predetermined orientation of the patient. All of this is done in accordance with a computer program. In this next orientation, another eye-movement record is created. Thus, an eye-movement record is created with collected eye-movement image data at each head orientation. After completion of the Dix-Hallpike Maneuver, the plurality of eye-movement records are collated and archived at the recorder. As indicated above, the recorder can be connected to an external data base which contains pertinent information on a plurality of specialists. Thereafter, in response to instructions from the patient, these eye-movement records are available for evaluation and use by selected specialists.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic diagram of a system for creating eye-movement records in accordance with the present invention;

FIG. 2 is a schematic of an environment for operation of the system of the present invention; and

FIG. 3 is an operational flow chart for the creation of eye-movement records in accordance with the present invention.

### DESCRIPTION OF THE PREFERRED EMBODIMENTS

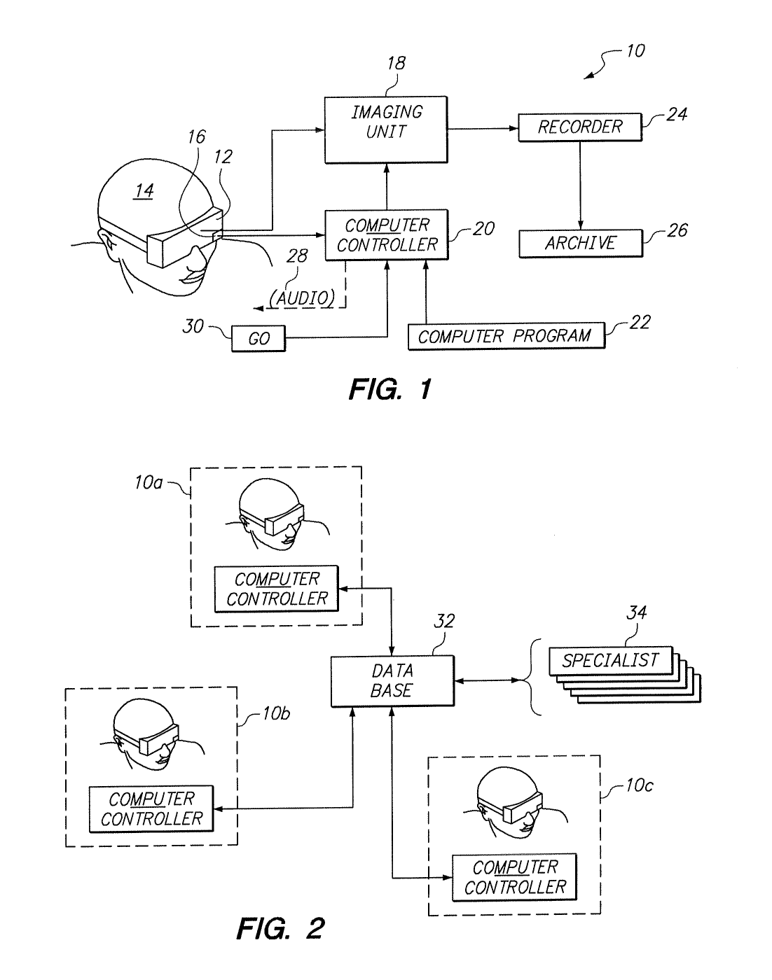

Referring initially to FIG. 1 a system for collecting eye-movement data, and for creating eye-movement records with the collected data, is shown and is generally designated 10. As shown, the system 10 includes goggles 12 that can be placed on the head of a patient 14. The import here is that the goggles 12 are to be held stationary on the head of the patient 14, for movement with the head of the patient 14. It is also shown in FIG. 1 that the goggles 12 include an indicator 16, and that they are connected with an imaging unit 18. For the present invention, the indicator 16 can be any type of inertial orienting device that is known in the pertinent art, and that is capable of identifying a spatial, three dimensional orientation for the head of patient 14. Also, it is to be appreciated that the imaging unit 18 includes a camera (not shown) for generating images of eye movements. For the system 10, the camera is mounted on the goggles 12.

Still referring to FIG. 1, it is shown that the system includes a computer/controller 20 that is mounted on the goggles 12 to essentially control the operation of system 10. This control is accomplished in accordance with a computer program 22. Further, FIG. 1 shows that a recorder 24 is electronically connected between the imaging unit 18 and an archive 26. Preferably, the recorder 24 is also mounted on the goggles 12. With this connection, the images of eye-movements (i.e. data) that are generated by the imaging unit 18, can be passed from the recorder 24 to the archive 26 where they will be collected, collated and stored for future use, as desired by the patient 14. It is also seen in FIG. 1 that an audio capability 28 is provided within the system 10 so that the computer/controller 20 can effectively give instructions to the patient 14. In response to these instructions, the patient 14 has the control of a “go” button 30 that is connected directly to the computer/controller 20. Use of the “go” button 30 is disclosed below, in detail, in conjunction with an operation of the system 10.

As envisioned for the present invention, the system 10 can be used in a clinical environment, in a physician's office, individually by the patient 14 at a remote site, or in any other appropriate outpatient location. Regardless where the system 10 is used, FIG. 2 indicates that the system 10 will normally be connected directly to a data base 32. The different systems 10a, 10b and 10c shown in FIG. 2 are only exemplary of such connections. FIG. 2 also shows that the data base 32 is accessible by a plurality of specialists 34, individually or collectively.

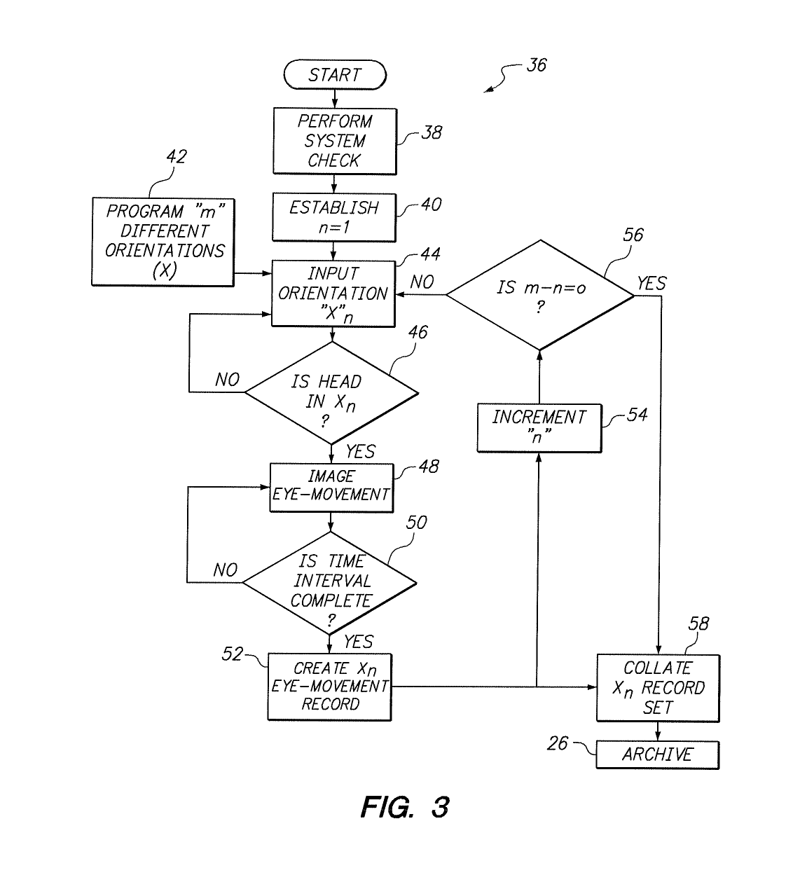

FIG. 3 presents a flow chart 36 for an operation of the system 10. In detail, after the computer/controller 20 has been programmed and turned “on”, block 38 indicates that the system 10 is to be checked. This involves ensuring that the goggles 12 are properly placed on the head of the patient 14, and that the system 10 is otherwise prepared for its operation. In this instance, block 40 requires the number of sequential eye-image iterations to be accomplished during an operation of the system 10 (i.e. n) be initially set with n=1. Block 42 of the flow chart 36 then requires the total number of iterations to be accomplished be set to the number m. For the present invention, m is the number of different head orientations x that are to be established in accordance with the Dix-Hallpike Maneuver. In this case, the Dix-Hallpike Maneuver requires head movements through a sequence of seven separate head orientations. Thus, m=7. In order, these orientations are:

1. patient sits with head centered;

2. patient sits with head 45 degrees to the right;

3. patient lays back with head 45 degrees to the right and held in approximately 20 degrees of extension;

4. patient sits back up with head centered;

5. patient sits with head 45 degrees to the left;

6. patient lays back with head 45 degrees to the left and held in approximately 20 degrees of extension; and

7. patient sits back up with head centered.

As indicated by block 44 of the operational flow chart 36, there is an operational cycle for each of the n different head orientations in the Dix-Hallpike Maneuver. In this context, the computer/controller 20 uses the audio 28 to tell the patient 14 what he/she is to do for each head orientation. For example, the initial operational cycle (x₁) requires the patient 14 move to a sitting position with his/her head centered. Block 44 specifies this action, and the patient 14 then complies with a prompt from the audio 28 and moves his/her head to the orientation prescribed for the operational cycle (i.e. x₁). Additionally, the computer program 22 requires confirmation that the patient's head is still, and is in the proper orientation in the video screen of the imaging unit 18 for proper video acquisition. At this point, the indicator 16 on goggles 12 will electronically inform the computer/controller 20 that this has been successfully accomplished (see inquiry block 46). Next, the computer/controller 20 informs the patient 14 that his/her head is properly oriented for x₁. When so informed, the patient 14 then presses on the “go” button 30 and the imaging unit 18 records eye-movement data (see action block 48) for a predetermined interval of time (e.g. ten seconds). After the time interval has expired (see inquiry block 50) the eye-movement data that is taken for x₁ is used to create an eye-movement record. Action block 52 then indicates this eye-movement data is processed by the recorder 24 and sent to the archive 26 for storage.

Action block 54 indicates that after an eye-movement record has been created for x₁, n is incremented to 2 (i.e. the system 10 will now function with x₂). Since m is 7 and n is 2, inquiry block 56 returns operation to action block 44 and the patient 14 will again be instructed by audio 28. This time, however, the patient 14 will be instructed to remain sitting and move his/her head 45° to the right. Thus, another operational cycle is initiated (i.e. x₂) and an eye-movement record will be created for x₂. According to the flow chart 36, these cycles of operation continue until x₇ has been accomplished and the Dix-Hallpike Maneuver has thereby been completed. At that time, action block 58 indicates that a complete set of eye-movement records are collated and passed to the archive 26. In addition to a video archive of eye-movement records, a dynamically positioned head movement data set is generated. This data set is collected and stored in each recording for subsequent evaluation and verification that the patient's head was still during the actual recording session.

Returning to FIG. 1, it will again be appreciated that the eye-movement records (i.e. x₁ through x₇) of many different patients 14 can be assembled at the centralized data base 32. Typically, however, these records will be de-identified before they are transferred to the data base 32. Thus, the confidentiality and use of the various eye-movement records are strictly maintained by the individual patient 14. Furthermore, once they are at the data base 32 the eye-movement records can be identified with a particular patient 14, only by instructions from the patient 14. Also, only with instructions from the patient 14 can eye-movement records be released from the data base 32 for use and evaluation by specialists 34.

In a preferred embodiment of the present invention, the indicator 16, the imaging unit 18 with its camera, the computer/controller 20 and the recorder 24 are integrated into a cohesive assembly. This assembly is then mounted (i.e. clipped) onto the goggles 12 and is used as a self-contained unit.

While the particular System and Method for Collecting Eye-Movement Data as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for collecting eye-movement data to create an eye-movement record for use as a diagnostic of balance disorders, wherein the eye-movements are responsive to a sequence of changes in head orientation, the system comprising:
an indicator mounted on the head of a patient for movement therewith, wherein the indicator generates position signals indicative of a spatial, three dimension orientation of the head;
an imaging unit positioned for movement with the head of the patient to create the eye-movement record for at least one eye of the patient, while the head of the patient is held substantially stationary for a preselected time interval, in a predetermined orientation;
a computer/controller connected to the indicator for notifying the patient when the head of the patient is in the predetermined orientation, and wherein the computer/controller is connected to the imaging unit for activating the imaging unit to create an eye-movement record in response to a “go” signal from the patient; and
a recorder connected to the imaging unit to archive the eye-movement record.

2. A system as recited in claim 1 further comprising:
a regional data base containing respectively pertinent information on a plurality of specialists; and
a central server incorporated with the recorder for forwarding a confidential eye-movement record to the data base for use by at least one selected specialist in response to instructions from the patient.

3. A system as recited in claim 2 wherein there are an m number of predetermined orientations to create m respective eye-movement records in accordance with a Dix-Hallpike Maneuver, and wherein the recorder is connected to the computer/controller to change the predetermined orientation of the patient in accordance with a computer program.

4. A system as recited in claim 3 wherein m is equal to seven (m=7).

5. A system as recited in claim 1 wherein the imaging unit includes a camera for creating images of the eye movements.

6. A system as recited in claim 5 wherein the system further comprises a pair of goggles, and wherein the indicator, the camera, and the computer/controller are mounted on the goggles.

7. A system as recited in claim 6 wherein the indicator, the imaging unit, the camera, the computer/controller and the recorder are integrated into a cohesive assembly and are collectively mounted as a self-contained unit on the goggles.

8. A system as recited in claim 1 further comprising a “go” button for use by the patient to activate the imaging unit in response to notification from the computer/controller that the head of the patient is in the predetermined orientation wherein the preselected time interval is less than ten seconds.

9. A method for collecting eye-movement data to create a confidential eye-movement record for use as a diagnostic of balance disorders, the method comprising the steps of:
mounting an orientation indicator on the head of a patient;
positioning an imaging unit, including a camera, on the head of the patient for movement therewith;
generating position signals with the orientation indicator, wherein the position signals are indicative of a spatial, three dimension orientation of the head;
notifying the patient when the head of the patient is in a predetermined orientation;
activating the imaging unit to collect eye-movement image data from at least one eye of the patient, while the head of the patient is held substantially stationary for a preselected time interval in the predetermined orientation; and
creating the confidential eye-movement record from the collected eye-movement image data with verification of the head of the patient being held substantially stationary during the activating step.

10. A method as recited in claim 9 further comprising the steps of:
de-identifying the eye-movement record to establish confidentiality and protect the identity of the patient; and
forwarding the de-identified eye-movement record to a regional specialist for evaluation and possible treatment in response to instructions from the patient.

11. A method as recited in claim 9 wherein the activating step is accomplished in response to a “go” signal from the patient, and wherein the “go” signal is initiated by the patient in response to the notifying step.

12. A method as recited in claim 9 further comprising the step of instructing the patient to move the head of the patient to a specific predetermined orientation.

13. A method as recited in claim 12 wherein the instructing step is accomplished prior to the generating step.

14. A method as recited in claim 12 wherein there are an m number of predetermined orientations to create m respective eye-movement records in accordance with a Dix-Hallpike Maneuver.

15. A method as recited in claim 14 wherein m is equal to seven (m=7).

16. A method as recited in claim 14 wherein the specific predetermined orientation of the head of the patient is selected, in order, as:
patient sits with head centered;
patient sits with head 45 degrees to the right;
patient lays back with head 45 degrees to the right and held in approximately 20 degrees of extension;
patient sits back up with head centered;
patient sits with head 45 degrees to the left;
patient lays back with head 45 degrees to the left and held in approximately 20 degrees of extension; and
patient sits back up with head centered.

17. A method as recited in claim 9 wherein the indicator and the camera are mounted on goggles.

18. A method as recited in claim 9 wherein the preselected time interval is less than ten seconds.

19. A method as recited in claim 9 wherein a plurality of eye-movement records are created and the method further comprises the steps of:
collating the plurality of eye-movement records;
archiving the plurality of eye-movement records;
maintaining a data base containing pertinent information on a plurality of specialists;
de-identifying each eye-movement record to maintain confidentiality and protect the identity of the respective patient; and
forwarding a de-identified eye-movement record to at least one selected specialist in the data base in response to instructions from the patient.

20. A computer program for collecting eye-movement data comprising program sections for respectively: generating position signals with an indicator, wherein the position signals are indicative of a spatial, three dimension orientation of the head; notifying the patient when the head of the patient is in a predetermined orientation; positioning an imaging unit, including a camera, for movement with the head of the patient; activating the imaging unit to create an eye-movement record of at least one eye of the patient, while the head of the patient is held substantially stationary for a preselected time interval in the predetermined orientation; collecting, collating, and archiving the plurality of eye-movement records; maintaining a data base containing pertinent information on each specialist in a plurality of specialists; de-identifying each eye-movement record to protect the identity of the respective patient; and forwarding a de-identified eye-movement record to at least one selected specialist in the data base in response to instructions from the patient.
